(19) United States
(12) Patent Application Publication
(10) Pub. No.: US 2023/0191112 A1
(43) Pub. Date: Jun. 22, 2023

(54) PORTABLE ELECTROSTIMULATION DEVICE

(71) Applicant: FLOWSTIM TECHNOLOGY, BIGANOS (FR)

(72) Inventor: Florent PAPILLAUD, BIGANOS (FR)

(21) Appl. No.: 17/999,125

(22) PCT Filed: May 18, 2021
(86) PCT No.: PCT/EP2021/063191

§ 371 Date: Nov. 17, 2022

(30) Foreign Application Priority Data
May 19, 2020 (FR) 2004999

(51) Int. Cl. A61N 1/04; A61N 1/36

(52) U.S. Cl. CPC A61N 1/0456; A61N 1/36007; A61N 1/0476; A61N 1/0484; A61N 1/36034

(57) ABSTRACT

The invention relates to a portable electrostimulation device comprising an ankle support and at least two electrodes connected electrically to a stimulator fixed to the ankle support, the electrodes being arranged on the inner face of the ankle support in such a way that the electrodes are in line with the tibial nerve of the patient when he or she puts on the ankle support, in which the ankle support has a first strap, a first fastening member arranged on the inner face of this first strap, and a second fastening member arranged on the outer face of the ankle support at the region of a first electrode, said first strap being intended to be wound around the foot of the patient in order to be turned back on the ankle support at the region of this first electrode.

The invention relates to the domain of electrostimulation for the treatment of incontinence. More precisely, the invention relates to a portable electrostimulation device.

It is known to treat patients for vesico-sphincter disorders, such as bladder hyperactivity, or anorectal disorders, notably anal incontinence, constipation, transit disorders, or functional colopathy, through electrostimulation, by applying an electric signal exhibiting certain specific parameters to the tibial nerve of this patient.

In a known manner, existing electrostimulation devices require the use of a specific stimulator, forcing the patient to isolate, position the electrodes on his or her ankle, then start electrostimulation and remain seated near the stimulator throughout the duration of this electrostimulation, i.e. approximately 20 minutes every day.

In order to alleviate this problem, there are also portable electrostimulation devices intended to be arranged on the ankle of the patient. However, the arrangement of these devices on the ankle of the patient remains complex and unergonomic. In particular, these portable electrostimulation devices do not allow the proper positioning of the electrodes in a simple manner, regardless of the ankle morphology of the patient.

In this context, there is thus a need for a portable electrostimulation device whose arrangement on the ankle of a patient is simple and ergonomic and which allows in particular the proper positioning of the electrodes in a simple manner. The present invention is positioned in this context and aims to meet this need.

To this end, the technical objective of the present invention is to provide a portable electrostimulation device comprising an ankle support and at least two electrodes connected electrically to a stimulator fixed to the ankle support, the stimulator being able to generate an electrostimulation signal and to transmit this electric signal to the electrodes, the ankle support being provided with an inner face and an outer face, the electrodes being arranged on the inner face of the ankle support in such a way that the electrodes are in line with the tibial nerve of the patient when he or she puts on the ankle support, in which the ankle support has a first strap, a first fastening member arranged on the inner face of this first strap, and a second fastening member arranged on the outer face of the ankle support at the region of a first electrode, said first strap being intended to be wound around the foot of the patient in order to be turned back on the ankle support at the region of this first electrode.

The electrodes are thus intended to come into contact with specific points on the ankle or foot of the patient when the patient puts the ankle support on his or her foot, and the use of a strap allows the ankle support to be attached or secured on the foot and thus contact between the electrodes and the ankle or the foot of the patient to be ensured. Furthermore, as the strap turns back to cover the ankle support at the region of the first electrode, it is ensured that the first fastening member will reach to the region of the second fastening member in order to reinforce this contact between the electrodes and the ankle of the patient. Thus, a robust, ergonomic, and easy-to-use electrostimulation device that guarantees constant contact between the electrodes and the foot or ankle of the patient can be guaranteed.

Advantageously, the electrodes are exclusively stimulation electrodes, and the stimulator is bereft of any function for the measurement of an electric potential that could be detected by these electrodes. Also advantageously, the stimulator is electrically connected to said electrodes so that each electrode simultaneously applies said electrostimulation signal generated by the stimulator to the patient.

In an embodiment of the invention, the first electrode is electrically connected to the second fastening member, and the stimulator comprises a third fastening member arranged to engage with the second fastening member and to transmit the electrostimulation signal to the first electrode via the second and third fastening member. Otherwise, the stimulator may comprise a fourth fastening member arranged to engage with the first fastening member when the first strap is turned back on the ankle support at the region of the first electrode. For example, the second and third fastening members may each comprise a male and respectively female part of a snap fastener. Alternatively, the second and third fastening members comprise mechanical holding sub-members intended to engage and electrical connection sub-members intended to engage. Preferably, the third fastening member is fixed on an anterior face of the stimulator intended to be face-to-face with the outer face of the ankle support, and the fourth fastening member is fixed on a posterior face of the stimulator, opposite the anterior face. Advantageously, the fourth fastening member comprises a loop-shaped textile band and the first fastening member comprises a hook-shaped textile band. It is thus understood that in this embodiment, the stimulator is fixed and electrically connected directly at the region of the first electrode, everything being held in place by the first strap.

Advantageously, the ankle support comprises a second strap, a fifth fastening member provided on the inner face of this second strap, and a sixth fastening member provided on the outer face of the ankle support at the region of a second electrode, said second strap being intended to be wound around the foot of the patient in order to be turned back on the ankle support at the region of this second electrode, and the fifth fastening member is arranged to engage with the sixth fastening member when the second strap is turned back on the ankle support at the region of the second electrode. Preferably, the device comprises an electrical connection member to electrically connect the first electrode to the second electrode. For example, the electrical connection member may comprise a cable or a bundle of cables arranged inside the ankle support.

In another embodiment of the invention, the first fastening member is arranged to engage with the second fastening member when the first strap is turned back on the ankle support at the region of the first electrode. Otherwise, the ankle support can comprise a second strap, a third fastening member provided on the inner face of this second strap, and a fourth fastening member provided on the outer face of the ankle support at the region of a second electrode, said second strap being intended to be wound around the foot of the patient to turn back on the ankle support at the region of this second electrode, and the third fastening member is arranged to engage with the fourth fastening member when the second strap is turned back on the ankle support at the region of the second electrode.

Advantageously, the stimulator comprises a fifth fastening member and the device comprises a sixth fastening member provided on the outer face of the ankle support between the first and second electrodes and arranged to engage with the fifth fastening member. Otherwise, the device may comprise electrical connection members to connect the stimulator to both the first and second electrodes.

According to an embodiment of the invention, the ankle support is a band formed of a single piece intended to be wound around the foot of the patient, one extremity of which band forms the first strap. Otherwise, the other extremity can form the second strap. If desired, the ankle support can be built using an elastomer, e.g. synthetic rubber. Advantageously, the stimulator exhibits a concave profile intended to mold to the shape of the foot. It can be foreseen to integrate the electrodes into the band, for example, by providing housings on the inner face of the band, in which these electrodes are installed so that their contact faces protrude from the inner face or are level with this inner face. Alternatively, the electrodes can be arranged on the inner face.

Advantageously, the stimulator comprises a wireless communication and control unit able to receive instructions to trigger the electrostimulation emitted by a distant electronic device and arranged to control, upon receipt of said instructions, the generation of said electrostimulation signal. The wireless unit may, for example, be able to receive and/or transmit data via a Bluetooth protocol and/or a Wi-Fi protocol.

Advantageously, the stimulator comprises a memory in which a plurality of sets of parameters of said electrostimulation signal are stored and wherein the wireless communication and control unit is arranged to select, upon receipt of the instructions, from within the memory a set of parameters based on said instructions and to control the generation of said electrostimulation signal exhibiting the parameters of the set of parameters selected

For example, the stimulator comprises a pulse width modulation electric current generator to generate said electrostimulation signal, this signal thus being composed of a series of electrical pulses according to a pulse frequency and a pulse duration (also called pulse length). For example, the generator can be arranged so that each pulse exhibits an amplitude below 100 mA, notably of approximately 15 mA, so that the pulse frequency of the pulse wave is between 1 and 120 Hz and so that the pulse length is between 50 and 600 μs. Otherwise, each set of parameters stored in the memory may contain a predetermined pulse frequency and a predetermined pulse length. Preferably, the stimulator may comprise an electrical power source, for example, a rechargeable battery, to electrically power the generator.

As an example, the following sets of parameters can be used, for example, to treat urinary incontinence: 10 Hz-200 μs; 14 Hz-210 μs; 15 Hz-150 μs; 50 Hz-200 μs. The following set of parameters can also be used to treat anal incontinence: 10 Hz-6000 μs.

Alternatively, the stimulator may comprise a direct current generator to generate said electrostimulation signal.

The present invention is now described through purely illustrative examples, which should in no way limit the range of the invention, and through the attached illustrations, in which:

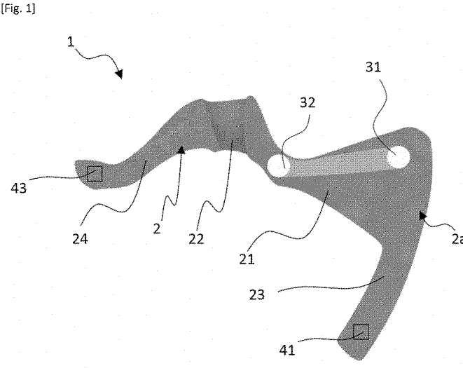

FIG. 1 is a schematic and partial representation of a portable electrostimulation device according to a first embodiment of the invention;

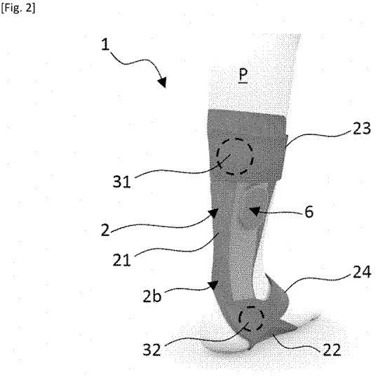

FIG. 2 is a schematic and partial representation of the device from [FIG. 1] arranged on the foot of a patient;

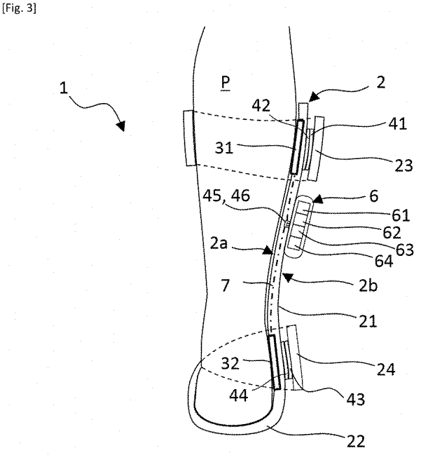

FIG. 3 is a schematic and partial representation of a cross-section of the device from [FIG. 2];

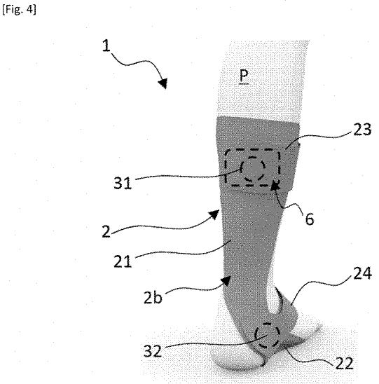

FIG. 4 is a schematic and partial representation of a portable electrostimulation device according to a second embodiment of the invention;

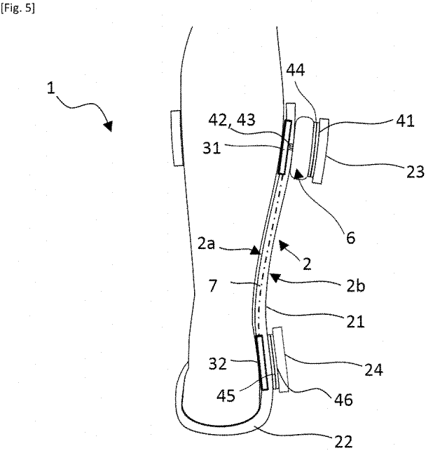

FIG. 5 is a schematic and partial representation of a cross-section of the device from [FIG. 4];

In the description that follows, elements that are identical in form or function appearing on different figures maintain the same reference numbers unless otherwise indicated.

FIG. 1 represents a portable electrostimulation device 1 according to a first embodiment of the invention. [FIG. 1] shows this device 1 flat, [FIG. 2] shows this device 1 arranged on the foot P of a patient, and [FIG. 3] shows a cross-section of the device 1 when it is arranged on the foot P of the patient as in [FIG. 2].

The portable electrostimulation device 1 comprises an ankle support 2 constructed in the form of a synthetic rubber band 2 comprising a central portion 21, from which a sole portion 22 extends. The band comprises a first extremity 23, extending from the central portion 21, opposite the sole portion 22, this first extremity 23 forming a first fastening strap 23. The band comprises a second extremity 24, extending from the sole portion 22, opposite the central portion 21, this second extremity 24 forming a second fastening strap 24. It is also foreseeable for the different portions of the band to have different thicknesses or to be constructed using different materials.

The ankle support 2 has an inner face 2a, intended to face the ankle P of the patient, and an outer face 2b, opposite the inner face 2a. [FIG. 1] thus shows the inner face 2a of the ankle support 2.

The device 1 comprises two electrodes 31 and 32, arranged on the inner face 2a of the ankle support 2, on the central portion 21. In the example described, housings are provided on the inner face 2a of the band 2 on the central portion 21, and each electrode 31, 32 is installed on one of the housings such that its contact face slightly protrudes from the inner face 2a.

The ankle support 2 comprises a set of fastening members allowing the device 1 to be held on the foot P of the patient, to ensure and reinforce contact between the electrodes 31 and 32 and the foot P of the patient. More specifically, the ankle support 2 comprises, on its inner surface 2a, at the region of each strap 23 and 24, first and third fastening members 41 and 43, constructed in this example in the form of hook-shaped textile bands glued or sewn on this inner surface 2a. The ankle support 2 also comprises, on its outer face 2b, at the region of each electrode 31 and 32, second and fourth fastening members 42 and 44, constructed in this example in the form of loop-shaped textile bands.

In order to put the device 1 on his or her foot P, as shown in [FIG. 2], the patient places the arch of his or her foot on the sole portion 22 of the ankle support 2 placed flat with its inner face 2a on the top, as shown in [FIG. 1]. He or she then lifts the central portion 21 to position it against his or her foot P, then winds the first strap 23 around his or her ankle until it folds back on the central portion 21 at the region of the electrode 31. The first fastening member 41 then engages with the second fastening member 42 to hold the ankle support 2 on and mold it to the foot P of the patient and press the electrode 31 against the skin. Likewise, the patient winds the second strap 24 around the back of the foot P until it folds back on the central portion 22 at the region of the electrode 32. The third fastening member 43 then engages with the fourth fastening member 44 to hold the ankle support 2 on and mold it to the foot P of the patient and press the electrode 32 against the skin. When the ankle support 2 is positioned on the foot P, as shown in [FIG. 2] and [FIG. 3], each electrode 31, 32 is then in line with the tibial nerve of the patient P such that an electrical signal transmitted to this electrode will stimulate this tibial nerve. In the example described, the shape of the ankle support 2 is defined such that the electrode 32 is a lower electrode, positioned approximately below the malleolus of the foot P and such that the electrode 31 is an upper electrode, positioned approximately above the lower electrode 32, for example, 15 cm above it.

The device 1 comprises a stimulator 6 able to generate an electrostimulation signal and connected electrically to the electrodes 31 and 32 so that each electrode 31 and 32 simultaneously applies to the patient this electrostimulation signal generated by the stimulator 6.

The stimulator 6 comprises a fifth fastening member 45 and the ankle support 2 comprises a sixth fastening member 46 provided on the outer face 2b of the ankle support 2, on the central portion 21, between the first and second electrodes 31, 32. The patient can thus fix the stimulator 6 on the ankle support 2 to form the device 1, by engaging the fifth fastening member 45 with the sixth fastening member 46. Advantageously, these fifth and sixth fastening members 45 and 46 are involved in the electrical connection of the stimulator 6 with the electrodes 31 and 32. For example, these fifth and sixth fastening members 45 and 46 can each comprise a male and respectively female part of a metallic snap fastener. In the example described, the sixth fastening member 46 is connected to each electrode 31, 32 by a cable 7 arranged inside the ankle support.

The stimulator 6 comprises a direct current pulse width modulation generator 61 to generate an electrostimulation signal in the form of a series of electrical pulses according to a given pulse frequency and a given pulse width. The stimulator 6 furthermore comprises a rechargeable battery 62 providing power to the generator 61 to allow the generation of the electrostimulation signal. The stimulator 6 furthermore comprises a memory 63 in which a plurality of sets of parameters of said electrostimulation signal are stored, each set containing a predetermined pulse frequency and a predetermined pulse width.

In order to be able to control the electrostimulation, the stimulator 6 is equipped with a Bluetooth communication and control unit 64 able to receive instructions to trigger the electrostimulation emitted by a distant electronic device, such as a smartphone, according to a desired program. Upon receipt of said instructions, the unit 64 selects within the memory 63 a set of parameters corresponding to the desired program contained in the instructions and controls the generation of an electrostimulation signal by the generator 61 exhibiting the pulse frequency and the pulse width defined in the set of parameters selected.

FIG. 4 represents a portable electrostimulation device 1 arranged on the foot P of a patient according to a second embodiment of the invention. [FIG. 5] shows a cross-section of the device 1 when it is arranged on the foot P of the patient as in [FIG. 4].

The device according to this second embodiment is in every way similar to that according to the first embodiment, with the exception of the fastening means 41 to 46 and the arrangement of the ankle support 2 on the foot of the patient.

In this embodiment, the ankle support 2 comprises, on its inner surface 2a, at the region of the strap 23, a first fastening member 41, and on its outer surface 2b, at the region of the first electrode 31, a second fastening member 42.

The first electrode 31 is connected electrically to the second fastening member 42. The stimulator 6 comprises a third fastening member 43, on its anterior surface, arranged to engage with the second fastening member 42 and to transmit the electrostimulation signal to the first electrode 31 via the second and third fastening members 42 and 43. In the example described, the second and third fastening members 42 and 43 each comprise a male and respectively female part of a snap fastener.

The stimulator 6 comprises a fourth fastening member 44, on its posterior surface. In the example described, the fourth fastening member 44 comprises a loop-shaped textile band and the first fastening member 41 comprises a hook-shaped textile band.

Similar to the first embodiment, the ankle support 2 comprises, on its inner surface 2a, at the region of the strap 24, a fifth fastening member 45, and on its outer surface 2b, at the region of the second electrode 32, a sixth fastening member 46. These fifth and sixth fastening members 45 and 46 are also made up of a hook-shaped textile band and a loop-shaped textile band, respectively.

To put the device 1 on his or her foot P, as shown in [FIG. 4], the patient places the arch of his or her foot on the sole portion 22 of the ankle support 2 placed flat with its inner face 2a on the top, as shown in [FIG. 4]. He or she then fixes the stimulator 6 on the ankle support 2 by engaging the third fastening member 43 with the second fastening member 42. He or she then lifts the central portion 21 to position it against his or her foot P, then winds the first strap 23 around his or her ankle until it folds back on the central portion 21 at the region of the electrode 31 and of the stimulator 6. The first fastening member 41 then engages with the fourth fastening member 44 to hold the ankle support 2 on and mold it to the foot P of the patient and press the stimulator 6 against electrode 31 and electrode 31 against the skin. Likewise, the patient winds the second strap 24 around the back of the foot P until it folds back on the central portion 22 at the region of the electrode 32. The fifth fastening member 45 then engages with the sixth fastening member 46 to hold the ankle support 2 on and mold it to the foot P of the patient and press the electrode 32 against the skin. It is thus understood that in this embodiment, the stimulator 6 is fixed and connected electrically directly at the region of the first electrode 31, everything being held on by the first strap 23.

The preceding description clearly explains how the invention allows the objectives for which it was created to be achieved, and notably by providing a portable electrostimulation device comprising an ankle support intended to bear the electrostimulation electrodes and equipped with one or several fastening straps intended to be wound around the foot or ankle of the patient to fold back on the ankle support at the region of one or each electrode. As the or each strap covers the ankle support at the region of an electrode, the contact is thus reinforced between this electrode and the ankle of the patient. Thus, a robust, ergonomic, and easy-to-use portable electrostimulation device that guarantees constant contact between the electrodes and the foot or ankle of the patient is provided.

In any case, the invention must not be limited to the embodiments specifically described in this document and particularly extends to all equivalent means and any technically operative combination of these means. In particular, other manners of fastening than those described can be foreseen, notably other types of fastening members. Other types of stimulator than the one described can also be foreseen, and notably stimulators able to generate continuous electrostimulation signals.

1. A portable electrostimulation device (1) comprising an ankle support (2) and at least two electrodes (31, 32) connected electrically to a stimulator (6) fixed to the ankle support, the stimulator being able to generate an electrostimulation signal and to transmit this electric signal to the electrodes, the ankle support being provided with an inner face (2a) and an outer face (2b), the electrodes being arranged on the inner face of the ankle support in such a way that the electrodes are in line with the tibial nerve of the patient when he or she puts on the ankle support, in which the ankle support has a first strap (23), a first fastening member (41) arranged on the inner face of this first strap, and a second fastening member (42) arranged on the outer face of the ankle support at the region of a first electrode (31), said first strap being intended to be wound around the foot (P) of the patient in order to be turned back on the ankle support at the region of this first electrode.

2. A device (1) according to the previous claim, wherein the first electrode (31) is connected electrically to the second fastening member (42), wherein the stimulator (6) comprises a third fastening member (43) arranged to engage with the second fastening member and to transmit the electrostimulation signal to the first electrode (31) via the second and third fastening members, and wherein the stimulator comprises a fourth fastening member (44) arranged to engage with the first fastening member (41) when the first strap (23) is turned back on the ankle support (2) at the region of the first electrode.

3. A device (1) according to the previous claim, wherein the ankle support comprises a second strap (24), a fifth fastening member (45) arranged on the inner face (2a) of this second strap, and a sixth fastening member (46) arranged on the outer face (2b) of the ankle support (2) at the region of a second electrode (32), said second strap being intended to be wound around the foot (P) of the patient in order to be turned back on the ankle support at the region of this second electrode, and wherein the fifth fastening member is arranged to engage with the sixth fastening member when the second strap is turned back on the ankle support at the region of the second electrode.

4. A device (1) according to claim 1, wherein the first fastening member (41) is arranged to engage with the second fastening member (42) when the first strap (23) is turned back on the ankle support (2) at the region of the first electrode (31), and wherein the ankle support comprises a second strap (24), a third fastening member (43) arranged on the inner face (2a) of this second strap, and a fourth fastening member (44) arranged on the outer face (2b) of the ankle support at the region of a second electrode (32), said second strap being intended to be wound around the foot (P) of the patient in order to be turned back on the ankle support at the region of this second electrode, and wherein the third fastening member is arranged to engage with the fourth fastening member when the second strap is turned back on the ankle support at the region of the second electrode.

5. A device (1) according to the previous claim, wherein the stimulator (6) comprises a fifth fastening member (45) and the device comprises a sixth fastening member (46) arranged on the outer face (2b) of the ankle support (2) between the first and second electrodes (31, 32) and arranged to engage with the fifth fastening member.

6. A device (1) according to the previous claims, wherein the ankle support (2) is a band made up of a single piece intended to be wound around the foot (P) of the patient, one extremity of which band forms the first strap (23).

7. A device (1) according to the previous claims, wherein the stimulator (6) comprises a wireless communication and control unit (64) able to receive instructions to trigger the electrostimulation emitted by a distant electronic device and arranged to control, upon receipt of the instructions, the generation of said electrostimulation signal.

8. A device (1) according to the previous claim, wherein the stimulator (6) comprises a memory (63) in which a plurality of sets of parameters of said electrostimulation signal are stored and wherein the wireless communication and control unit (64) is arranged to select, upon receipt of the instructions, from within the memory a set of parameters based on said instructions and to control the generation of said electrostimulation signal exhibiting the parameters of the set of parameters selected.

9. A device (1) according to the previous claim, wherein the stimulator (6) comprises a pulse width modulation electric signal generator (61), and wherein each set of parameters stored in the memory (63) contains a predetermined pulse frequency and a predetermined pulse width.
